(19) United States
(12) Patent Application Publication
(10) Pub. No.: US 2014/0031678 A1
(43) Pub. Date: Jan. 30, 2014

(54) METHOD FOR DRIVING WAVELENGTH-SWEPT LIGHT SOURCE

(71) Applicant: CANON KABUSHIKI KAISHA, Tokyo (JP)

(72) Inventor: Tomohiro Yamada, Yokohama-shi (JP)

(73) Assignee: CANON KABUSHIKI KAISHA, Tokyo (JP)

(21) Appl. No.: 13/947,317

(22) Filed: Jul. 22, 2013

(30) Foreign Application Priority Data
Jul. 25, 2012 (JP) 2012-164477

(51) Int. Cl. A61B 5/00; H01S 3/10

(52) U.S. Cl. CPC A61B 5/0066; H01S 3/10038
USPC 600/425; 372/26

(57) ABSTRACT

Disclosed is a method for driving a wavelength-swept light source that includes an optical resonator in which an optical gain medium for emitting light, a wavelength selection element for selecting a wavelength of light emitted from the optical resonator, and an optical intensity modulation element for modulating intensity of light within the optical resonator are arranged and that is configured to sweep the wavelength of the emitted light. The method includes driving the optical intensity modulation element and the wavelength selection element in synchronization with each other to keep an instantaneous spectral width of the emitted lights constant.

### BACKGROUND OF THE INVENTION

1. Field of the Invention

The present disclosure relates to a method for driving a wavelength-swept light source.

2. Description of the Related Art

In techniques for varying an oscillation wavelength of a light source, or in particular, of a laser light source, there is a strong demand for achieving both high-speed wavelength sweeping and a narrow linewidth.

In swept source optical coherence tomography (SS-OCT), spectral interference is used to obtain depth information. In SS-OCT, a broad interference spectrum is obtained using a wavelength-swept light source. Since a spectrometer is not used in SS-OCT, the loss in the quantity of light is small, and thus an image with a high signal-to-noise (SN) ratio is expected.

When constructing a medical imaging apparatus to which the SS-OCT technique is applied, as the sweeping speed is higher, the time it takes to obtain an image can be reduced, and thus such a medical imaging apparatus is suited to observing living biological tissues.

In an SS-OCT apparatus (swept source optical coherence tomographic imaging apparatus), a tomographic structure of an object is obtained using optical interference, and thus control of the coherence length of a light source is important.

Japanese Patent Laid-Open No. 2010-62426 discusses a wavelength-swept laser light source that can be applied to an SS-OCT apparatus and that includes an optical resonator, a wavelength tunable optical filter, and a current controller that varies the level of current injected into a gain medium.

### SUMMARY OF THE INVENTION

If the coherence length of a light source included in an SS-OCT apparatus varies during wavelength sweeping, the amplitude of an interference light component in a signal obtained by the OCT apparatus fluctuates. This means that an intensity variation is superimposed onto an interference signal, and as a result, noise is added to an obtained OCT image (tomographic image).

Accordingly, there has been a desire to suppress a variation in the coherence length of the light source during high-speed wavelength sweeping.

Japanese Patent Laid-Open No. 2010-62426 discusses a method for controlling the quantity of light from the light source to a certain value or less. However, this is for preventing a breakdown of a gain medium caused by current injection, and the fluctuation in the coherence length, which the present inventors see as an issue, is not discussed.

According to an aspect of the present invention, a method for driving a wavelength-swept light source that includes an optical resonator within which an optical gain medium for emitting light, a wavelength selection element for selecting a wavelength of light emitted from the optical resonator, and an optical intensity modulation element for modulating intensity of light within the optical resonator are arranged and that is configured to sweep a wavelength of the emitted light includes driving the optical intensity modulation element and the wavelength selection element in synchronization with each other to keep an instantaneous spectral width of the emitted light constant.

According to the method for driving the wavelength-swept light source of the above aspect of the present invention, the instantaneous spectral width of the light emitted from the light source is kept constant. Accordingly, with an optical coherence tomographic imaging method that employs the method of the above aspect of the present invention, the fluctuation in the coherence length of the wavelength-swept light source during wavelength sweeping is suppressed, and a tomographic image (OCT image) with less noise can be obtained.

Further features of the present invention will become apparent from the following description of exemplary embodiments with reference to the attached drawings.

### BRIEF DESCRIPTION OF THE DRAWINGS

FIG. 1 is a schematic diagram illustrating an example of a wavelength-swept light source to which an exemplary embodiment of the present invention can be applied.

FIG. 2 is a diagram for describing the exemplary embodiment of the present invention.

FIG. 3 is another diagram for describing the exemplary embodiment of the present invention.

FIGS. 4A and 4B are diagrams for describing a first exemplary embodiment of the present invention.

FIG. 5 is a diagram for describing wavelength sweeping in the first exemplary embodiment of the present invention.

FIG. 6 is a diagram for describing a wavelength sweeping spectrum in the first exemplary embodiment of the present invention.

FIG. 7 is a diagram for describing a spectrum in the first exemplary embodiment of the present invention.

FIG. 8 is a diagram for describing a second exemplary embodiment of the present invention.

FIG. 9 is another diagram for describing the second exemplary embodiment of the present invention.

FIG. 10 is a diagram for describing an interference spectrum in the first exemplary embodiment of the present invention.

### DESCRIPTION OF THE EMBODIMENTS

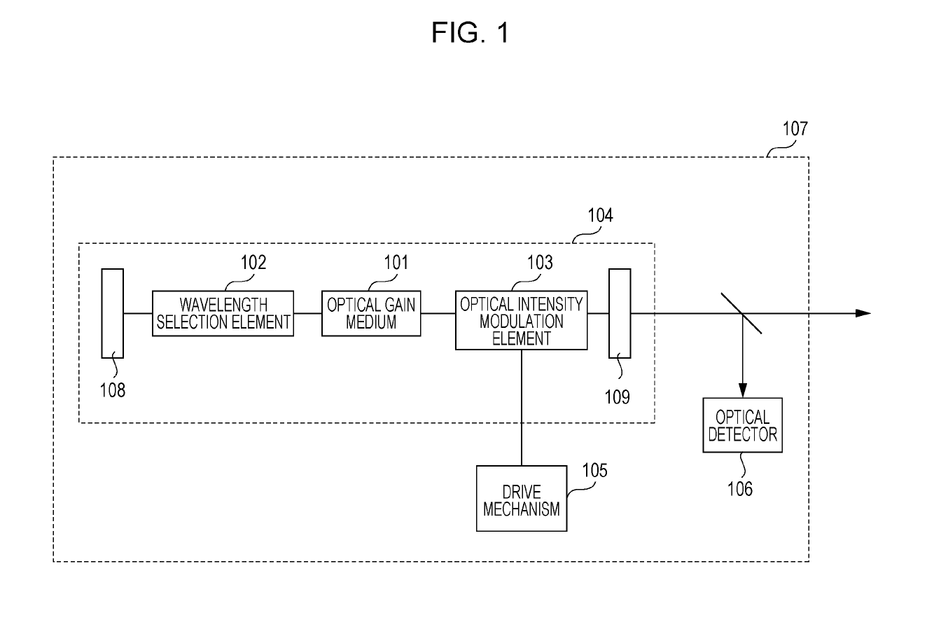

Hereinafter, an exemplary embodiment of the present invention will be described with reference to FIG. 1.

FIG. 1 is a schematic diagram illustrating an example of a wavelength-swept light source to which the exemplary embodiment of the present invention can be applied.

With reference to FIG. 1, mirrors 108 and 109 are included in an optical resonator 104, and an optical gain medium 101, a wavelength selection element 102, and an optical intensity modulation element 103 are disposed within the optical resonator 104. The optical gain medium 101 emits light, the wavelength selection element 102 selects a wavelength of light emitted from the optical resonator 104, and the optical intensity modulation element 103 modulates the intensity of light within the optical resonator 104. The optical intensity modulation element 103 is connected to a drive mechanism 105.

A change in the intensity of light emitted from the optical resonator 104 is monitored by an optical detector 106. The above-described components collectively constitute a light source unit 107 in FIG. 1.

In the exemplary embodiment of the present invention, for example, a tunable Fabry-Perot filter, a filter that disperses light with a diffraction grating and selects a specific wavelength, a high-speed wavelength tunable filter that is based on the vernier effect, or the like can suitably be used as the wavelength selection element 102. Further, it is preferable that the selectable wavelength bandwidth of the wavelength selection element 102 can be varied.

As the optical intensity modulation element 103, a transmittance tunable optical filter that is formed of a Pockels cell, a Kerr cell, or another electro-optical modulator (EOM), a liquid crystal, or the like or an acousto-optical modulator (AOM), for example, can be used.

Furthermore, various other elements such as an element that modulates the intensity of light passing therethrough by varying reflectance using a magneto-optical effect or an element that can vary the intensity of optical absorption through an electroabsorption effect can be used as well.

Alternatively, a glass plate whose angle relative to an optical path changes may be inserted. Another mechanism may be one with which coupling efficiency is varied by adjusting alignment of a half-silvered mirror or another optical component to allow the resonator loss to vary.

The optical detector 106 may be a simple photodiode. Alternatively, the optical detector 106 may be an element with photoelectromotive force or may employ another optical detection method.

Examples of the optical gain medium 101 include a semiconductor optical amplifier (SOA). A semiconductor optical amplifier is preferable since it has a broad gain bandwidth, is small, and can be controlled at high speed.

A compound semiconductor for forming a typical semiconductor laser can be used as a material for forming the semiconductor optical amplifier, and specific examples may include GaAs-based, InGaAs-based, InAsP-based, GaAlSb-based, GaAsP-based, AlGaAs-based, and GaN-based compound semiconductors. The semiconductor optical amplifier can appropriately be selected in accordance with the use of the light source, and for example, a semiconductor optical amplifier with the central wavelength of the gain bandwidth of 840 nm, 1060 nm, 1300 nm, or 1550 nm can be used.

Although the light source unit 107 illustrated in FIG. 1 includes a linear optical resonator, the type of the optical resonator is not limited thereto, and a ring resonator or a σ resonator may be used as well.

In the SS-OCT apparatus, interference-based measurement is carried out using light emitted from the light source unit 107.

The optical resonator 104 emits laser light. Here, since an external resonator type laser has a greater resonator length, its free spectral range (FSR) is small, and thus typically an external resonator type laser oscillates in multiple modes with a plurality of longitudinal modes.

When a multimode laser is used in a light source unit of an OCT apparatus, an instantaneous linewidth (i.e., instantaneous oscillation spectral width) for determining a measurable depth corresponds to the linewidth of the envelope of a spectrum of the plurality of longitudinal modes oscillating in the multimode.

In the description to follow, this instantaneous spectral width (i.e., instantaneous linewidth) is also referred to as a multimode linewidth.

In an SS-OCT apparatus, when an instantaneous spectral width (instantaneous linewidth) varies and the coherence length varies as well, interference intensity of reflected light from a sample (i.e., object to be measured) and reference light varies.

Accordingly, amplitude modulation of an interference signal that corresponds to a variation in the coherence length is superimposed onto an interference spectrum that is obtained by the SS-OCT apparatus.

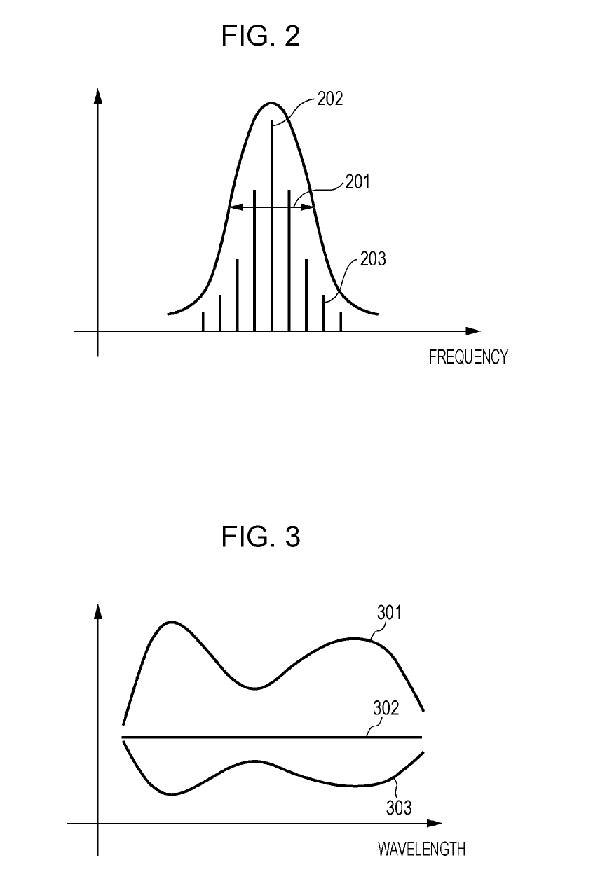

The amplitude modulation of the interference signal is reflected as noise on an OCT image (tomographic image) to be obtained in the end, and thus it is understood that suppressing a variation in the coherence length during wavelength sweeping presents an advantage in a light source to be used in the SS-OCT apparatus. This point will be described, hereinafter, with reference to FIG. 2.

Typically, a multimode linewidth 201 becomes narrower as the gain of an optical gain medium becomes larger, that is, as a net gain in a resonator becomes larger.

This is because a central mode m0 202 among the multiple modes has a greater gain than an adjacent side mode ms 203 through lasing, and a mode with higher photon density oscillates more intensely.

Accordingly, as the net gain is increased, the multimode linewidth becomes narrower, and the coherence length increases.

However, if the net gain increases excessively, the photon density of the central mode m0 202 does not increase in proportion to the amount of current injected into the optical gain medium, due to various factors such as hole burning, influence of heat, and gain saturation.

Then, an excess gain is distributed to the side mode ms 203, and as a result, the multimode linewidth is broadened.

Accordingly, it is indicated that the instantaneous spectral width takes the minimum value when the net gain is at a certain level. The net gain within the optical resonator is influenced by the coherence length, as described above. Therefore, the coherence length of the light source can be controlled by controlling the net gain within the resonator. In addition, there is an optimal value for maximizing the coherence length.

Furthermore, if the net gain during wavelength sweeping is controlled to stay constant, a variation of the coherence length during sweeping can be suppressed.

The net gain within the resonator is determined by a product of the gain of the optical gain medium itself and the loss caused by various optical elements provided within the resonator.

When a wavelength sweeping operation is carried out while driving the optical gain medium with a constant current, the wavelength sweeping spectrum reflects the net gain spectral profile.

Accordingly, as a method for suppressing a variation in the net gain during wavelength sweeping to suppress a variation in the coherence length for each wavelength in the OCT apparatus, a method can be considered in which the net gain is kept constant while sweeping the wavelength.

Control to keep the net gain constant corresponds specifically to keeping light emission intensity during wavelength sweeping constant.

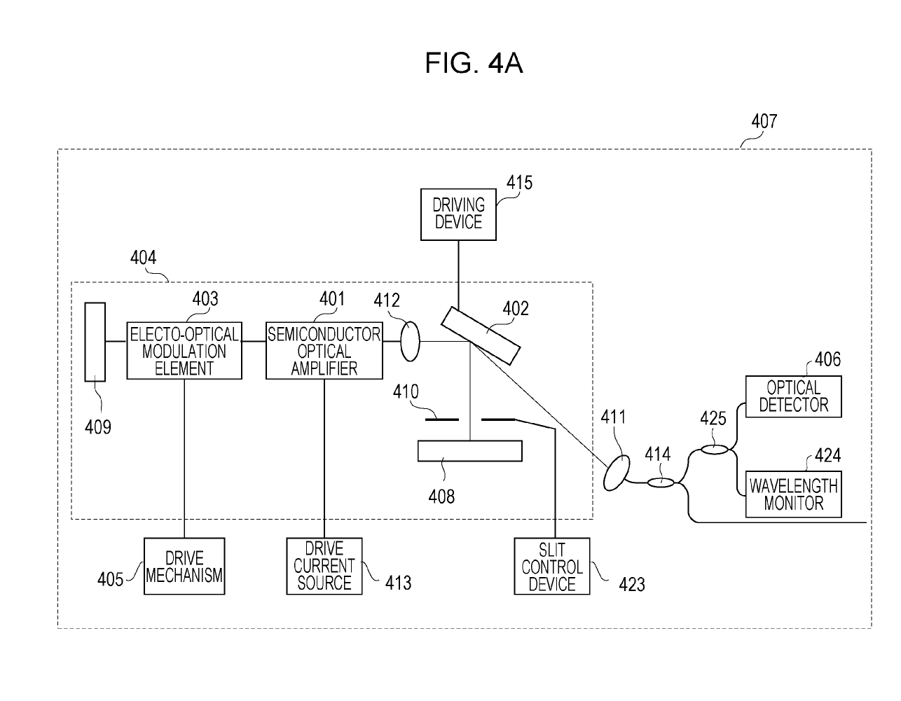

Here, a method for controlling a net gain spectrum to stay constant with respect to the wavelength sweeping operation will be described with reference to FIG. 3.

In FIG. 3, a net gain spectrum of the optical resonator when a loss spectrum of the optical resonator is not changed during wavelength sweeping is represented as a gain spectrum a(λ)_301. Then, a desired net gain spectrum b(λ)_302 is obtained as a result of changing the loss spectrum. That is, the gain spectrum b(λ)_302 represents a spectrum of light when the optical intensity is modulated by an optical intensity modulation element.

Further, a value of transmittance or reflectance of the optical intensity modulation element at an instantaneous light emission (oscillation) wavelength λ is represented as c(λ)_303. Here, when a transmissive-type optical intensity modulation element is used, its transmittance is used, and when a reflective-type optical intensity modulation element is used, its reflectance is used. The curve designated by 303 represents an envelope, and c(λ) can be taken as a spectrum of the optical intensity modulation element with respect to each wavelength λ when the optical intensity modulation element is driven to sweep the wavelength.

For example, if there is no loss caused by the optical intensity modulation element for all of the wavelengths λ, c(λ) is considered to be 1.

When there is a loss caused by the optical intensity modulation element, the net gain spectrum b(λ) of the optical resonator is a product of the original net gain spectrum at each wavelength and the transmittance or the reflectance of the optical intensity modulation element, as expressed through the following formula (1).

b(λ)=(1/α)×a(λ)×c(λ) Formula (1)

Here, α is a positive real number.

That is, in order to keep b(λ) constant regardless of the wavelength, the optical intensity modulation element may be driven so as to cancel out the wavelength dependence of the original net gain spectrum a(λ). By driving the optical intensity modulation element within the optical resonator during wavelength sweeping such that c(λ) satisfies the following formula (2), the light emission intensity can be kept constant, and thus the instantaneous linewidth of the light source can be controlled to stay constant.

c(λ)=α×b(λ)/a(λ) Formula (2)

In addition, in addition to carrying out the control to keep the light emission intensity constant, it is preferable that light emission intensity that corresponds to a net gain at which the coherence length is at the maximum length is obtained in advance and light emission intensity during actual optical coherence tomographic imaging is kept constant at that obtained light emission intensity.

The above can be achieved by introducing light from a light source to which the control method of the exemplary embodiment of the present invention is applied to a simple reference sample such as an interferometer with a variable optical path length difference and by observing the dependence of the interference intensity on the optical path length difference. For example, an optical path length difference at which the interference intensity is half of the interference intensity when the optical path length difference is 0 can be evaluated as the coherence length.

Then, it is preferable to search for a light source intensity at which the greatest coherence length is obtained by varying light source intensity so as to be kept constant. Note that it may also be possible to control the drive current of the optical amplifier in order to keep the net gain constant. However, in reality, it is difficult to precisely modulate the net gain within the resonator at high speed only by controlling the drive current of the optical amplifier in high-speed wavelength sweeping with an A-scan rate of a few hundred kHz or higher.

Further, in a semiconductor optical amplifier (optical gain medium), a variation in carrier density caused by drive current modulation or a fluctuation in an refractive index caused by a temperature variation may prevent a laser from oscillating stably, and thus a large variation in current injected into the optical amplifier is not preferable.

However, it is preferable to simultaneously control the drive current of the optical amplifier and modulate the optical intensity modulation element. That is, it is preferable to simultaneously control driving of the optical gain medium and the optical intensity modulation element.

For example, even when high-speed and precise control of the net gain within the resonator is difficult only by controlling the drive current of the optical amplifier as described above, it is possible and also preferable to cause the optical intensity modulation element to fine-tune the net gain while the control of the drive current of the gain medium is set to a gradual (low-frequency) input.

Through such control, a broadly flat net gain spectrum can be more easily realized. For example, in order to secure a broad oscillation bandwidth, large current needs to be supplied to the optical amplifier. Through this, a broader wavelength band with a gain that surpasses the loss within the resonator can be secured.

Accordingly, the light emission intensity at a wavelength at which the maximum gain is obtained (λmax) is extremely enhanced. That is, in order to obtain a broadly flat net gain, the optical absorption or loss of the optical intensity modulation element needs to be large at the wavelength λmax.

This requires the optical intensity modulation element to have a large dynamic range or a capability of absorbing a large quantity of light, and a heavy performance load is imposed on the optical intensity modulation element in order to satisfy such performance requirements.

Accordingly, it is extremely preferable to combine control of the low-frequency drive current of the optical gain medium in which the drive current of the optical gain medium is increased in a wavelength band that corresponds to a tail of the gain and the drive current is decreased in a wavelength band around the center of the gain bandwidth with correction of a high-frequency component using a high-speed optical intensity modulation element.

Through such control, performance requirements such as the large dynamic range and durability against heat generated by light absorption in the high-speed optical intensity modulation element can be eased.

Here, although a method that employs a gain flattening filter (GFF) can be considered as well, a GFF has a fixed transmittance profile and cannot accommodate the intensity variations caused by various external disturbances that are imposed on the resonator.

Furthermore, when the light emission intensity is to be changed, the drive current of the gain medium needs to be changed as well. However, as the drive current amount of the gain medium changes, the gain profile thereof changes as well, and thus there is still an issue that cannot be taken care of with the GFF.

Accordingly, the method of the exemplary embodiment of the present invention for controlling the instantaneous oscillation spectral width using the optical intensity modulation element that is free from the above issues is advantageous.

In addition, when controlling the instantaneous spectral width to stay constant, aside from controlling the quantity of emitted light to stay constant as described above, it is also preferable to drive the optical intensity modulation element such that the coherence length stays constant while directly monitoring the coherence length of the light source.

For example, as in the above-described coherence length evaluation, there exists a method in which a sample with a known optical path length (e.g., a simple interferometer, a Fabry-Perot resonator, or the like) is irradiated with light from a wavelength-swept light source and an interference spectrum is obtained while varying the optical path length difference.

By extracting an amplitude component of an interference signal at a given wavelength from interference spectra obtained with respect to various optical path lengths and evaluating the distance dependence thereof, the coherence length can be monitored as an optical path length difference at which the amplitude of the interference signal at the given wavelength is ½.

Alternatively, it is also possible to find a spectral shape at a given wavelength by measuring the quantity of light from a light source through a band-pass filter or a notch filter with known spectral characteristics while sweeping the wavelength. Furthermore, use of a wavelength selection element with a variable selectable wavelength range is also preferable in controlling the coherence length to stay constant.

The multimode linewidth of the light source depends on the net gain within the resonator as described above and also depends on the wavelength selection spectral shape of the wavelength selection element.

This is because the ratio of the net gain at the central mode m0 and the net gain at the side mode ms, which determine the spectral shape of the multimode oscillation, depends on the spectral shape of the stated wavelength selection element.

For the sake of simplicity, the wavelength selection spectral shape is assumed to be Gaussian. By changing the full width at half maximum of this Gaussian spectrum, the multimode linewidth can be changed as well. Specifically, in order to make the multimode linewidth narrower, the wavelength selection spectral shape may be made narrower.

Accordingly, it is also preferable to monitor the coherence length of the light source as described above and to vary the width of the wavelength selection spectral shape so that the coherence length stays constant.

As a wavelength selection element that is capable of such control, there is, for example, one having a configuration in which light is dispersed by a diffraction grating and part of the light at a certain wavelength is extracted through a narrow slit.

In such an element, for example, changing the slit width allows the wavelength selection width to be changed. As the slit is made narrower, the wavelength selection width becomes narrower as well.

Alternatively, while keeping the slit width constant, the width, in the lattice vector direction, of light flux to be incident on the diffraction grating may be changed. In this case, as the light flux to be incident on the diffraction grating becomes wider, the wavelength selection width becomes narrower.

In the description herein, although a wavelength selection element formed of a diffraction grating is used, the wavelength selection element is not limited thereto, and a wavelength selection element formed of a prism or the like may instead be used.

Here, in order to increase the observation depth of the OCT apparatus, a continuous wave light source is more preferable than a pulse light source. This is because a short pulse duration leads to a broad spectral bandwidth, and in turn the coherence length is reduced.

Accordingly, regardless of whether the sweeping method of the wavelength-swept light source is continuous wavelength sweeping or step-wise wavelength sweeping, the continuous wave light source is preferable as an OCT light source.

The optical intensity modulation element is preferably disposed at a position other than a position between the gain medium and the wavelength selection element.

Typically, a wavelength-swept light source oscillates in multiple modes while sweeping the wavelength at high speed.

Accordingly, it is preferable that the multimode oscillation rises quickly and reaches a stable oscillation state as quickly as possible. For that reason, it is desirable that more light at a wavelength narrowly selected by the wavelength selection element returns to the optical gain medium and is amplified therein.

Further, since it is not possible to suppress the loss of the optical intensity caused by the optical intensity modulation element to zero, some loss is inevitable.

Accordingly, in view of the above, it is not preferable that the optical intensity modulation element be disposed in an optical path through which light extracted by the wavelength selection element returns to the optical gain medium.

Furthermore, in the SS-OCT apparatus, the wavelength sweeping bandwidth affects the depth-wise resolution.

Specifically, if the wavelength sweeping spectral shape is Gaussian, the relationship expressed by the following formula (3) holds true.

$$Δ z = 2 \frac{ln 2}{π} \times \frac{{λ}_{0}^{2}}{Δ λ} \qquad Formula ( 3 )$$

In the above formula, λ₀ is the central wavelength of wavelength sweeping, Δλ is the width of the wavelength sweeping bandwidth, and the Δz is the depth-wise resolution.

Accordingly, in order to satisfy the desired depth-wise resolution Δz, the wavelength sweeping bandwidth Δλ needs to be a value that is equal to or greater than a value expressed through the following formula (4).

$$Δ λ = 2 \frac{ln 2}{π} \times \frac{{λ}_{0}^{2}}{Δ z} \qquad Formula ( 4 )$$

That is, the wavelength sweeping bandwidth is preferably set to a value that is equal to or greater than 2×ln(2)/π×λ₀²/Δz, in which Δz is the depth-wise resolution in the optical coherence tomographic imaging and λ₀ is the central wavelength of the wavelength band in which the wavelength is swept.

An exemplary embodiment of the present invention encompasses an optical coherence tomographic imaging method.

In an optical coherence tomographic imaging method according to an exemplary embodiment of the present invention, light emitted from a light source unit is split, and the split rays are radiated onto a sample and a reference reflection portion, respectively. Then, a tomographic image of the sample is obtained based on interference light in which reflection light from the sample and reflection light from the reference reflection portion interfere with each other.

In the stated imaging method, a wavelength-swept light source that is configured to sweep the wavelength of light to be emitted therefrom and that includes, as a light source unit, an optical resonator in which an optical gain medium for emitting light, a wavelength selection element for selecting a wavelength of light emitted from the optical resonator, and an optical intensity modulation element for modulating intensity of light within the optical resonator are arranged is used. In the stated imaging method, the optical intensity modulation element and the wavelength selection element are driven in synchronization with each other, and thus an instantaneous spectral width of the emitted light is kept constant.

With a driving method and the imaging method of the exemplary embodiment of the present invention, an interference spectrum is obtained with a light source that realizes a long coherence length over a wide range. Thereafter, data is re-sampled at equal wave number intervals, and the data is then subjected to an appropriate window function such as Gaussian to perform a Fourier transform to obtain a tomographic image. In this case, it is necessary to keep the light source intensity constant to suppress a fluctuation in the coherence length within a bandwidth that is equal to or greater than a wavelength width of the wavelength band Δλ necessary for the Gaussian spectrum. With the stated imaging method, the fluctuation in the coherence length during an A-scan can be suppressed, and an OCT image with less noise can be obtained. Hereinafter, the present invention will be described in further detail with specific exemplary embodiments.

### First Exemplary Embodiment

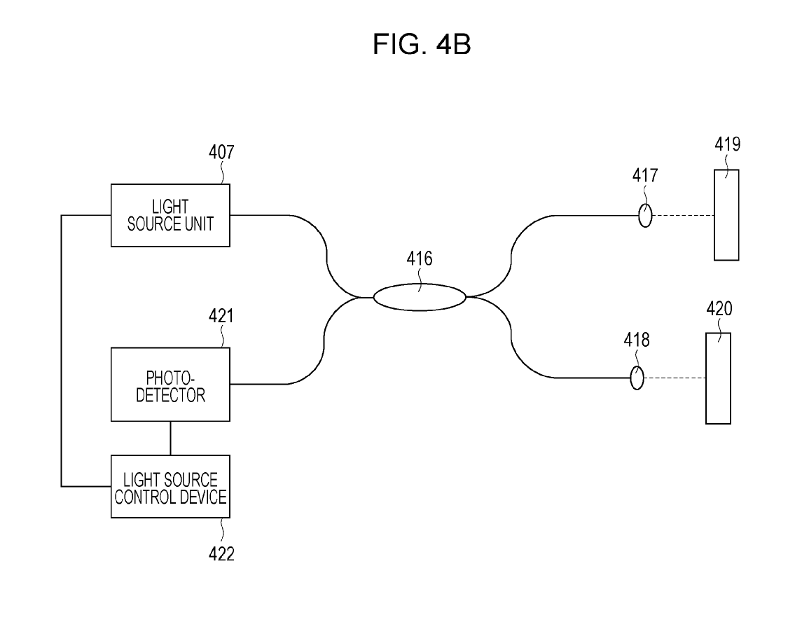

FIG. 4A is a schematic diagram illustrating a light source to which a method for driving a wavelength-swept light source according to a first exemplary embodiment is applied.

The light source illustrated in FIG. 4A includes an optical resonator 404 that includes a semiconductor optical amplifier 401, a diffraction grating 402, and an electro-optical modulation element 403 formed of a Pockels cell. The light source further includes a slit 410 and mirrors 408 and 409, and the slit width of the slit 410 is controlled by a slit control device 423.

Collimators 411 and 412 and a drive current source 413 for the semiconductor optical amplifier 401 are also provided. A driving device 415 controls the deflection angle, the amplitude, and the frequency of the diffraction grating 402.

The electro-optical modulation element 403 is connected to a drive mechanism 405.

Light emitted from the optical resonator 404 is guided to an optical detector 406 through optical couplers 414 and 425 and a change in the optical intensity of the light is measured by the optical detector 406. A wavelength monitor 424 that includes a k-clock system is also provided to obtain signals at equal frequency intervals in a wavelength sweeping state. The above-described components collectively constitute a light source unit 407.

In the SS-OCT apparatus, interference-based measurement is carried out using light emitted from the light source unit 407.

In an OCT apparatus with an assumed depth-wise resolution of 3 μm, if the wavelength sweeping spectrum is Gaussian with a central wavelength of 840 nm, its full width at half maximum needs to be approximately 80 nm.

Accordingly, in the first exemplary embodiment, control is carried out to suppress a fluctuation in the coherence length across the wavelength band of 80 nm or more.

The light source unit 407 sweeps the wavelength from 800 nm to 880 nm. The diffraction grating 402 deflects the angle of the grating surface at a frequency of 25 kHz. The diffraction grating 402 has line density of 600 lines per millimeter, and its deflection angle is approximately ±4 degrees.

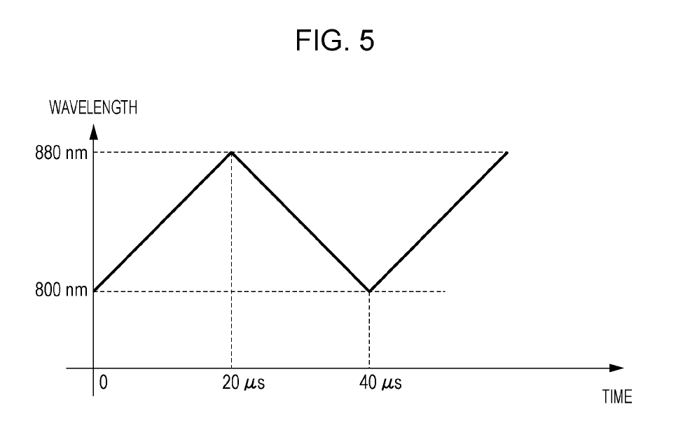

A schematic diagram of wavelength sweeping is illustrated in FIG. 5.

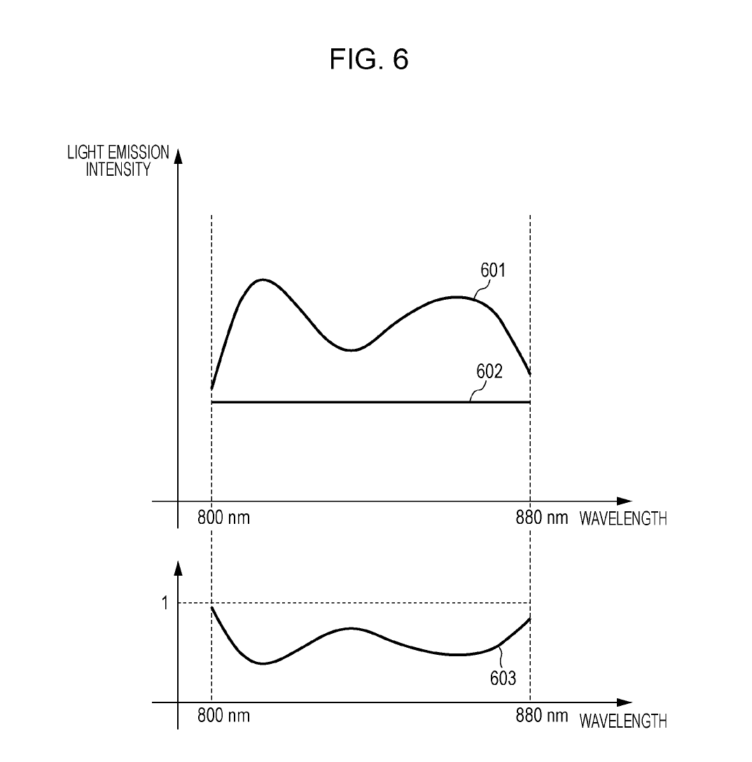

A wavelength sweeping spectrum 601 while the transmittance of the electro-optical modulation element 403 is kept constant is illustrated in FIG. 6.

The transmittance of the electro-optical modulation element 403 is swept along a curve 603 from a shorter wavelength side to a longer wavelength side and from a longer wavelength side to a shorter wavelength side in synchronization with the wavelength sweeping frequency of 25 kHz, and thus a light emission spectrum 602 that is flat across this wavelength band is obtained. Through such control, the fluctuation in the coherence length during wavelength sweeping can be suppressed. Further, if the wavelength dependence of the light emission intensity of the light source is large, not only the electro-optical modulation element 403 but also the drive current of the semiconductor optical amplifier 401 may be modulated as well. In addition, it is also possible to directly monitor the instantaneous linewidth during wavelength sweeping.

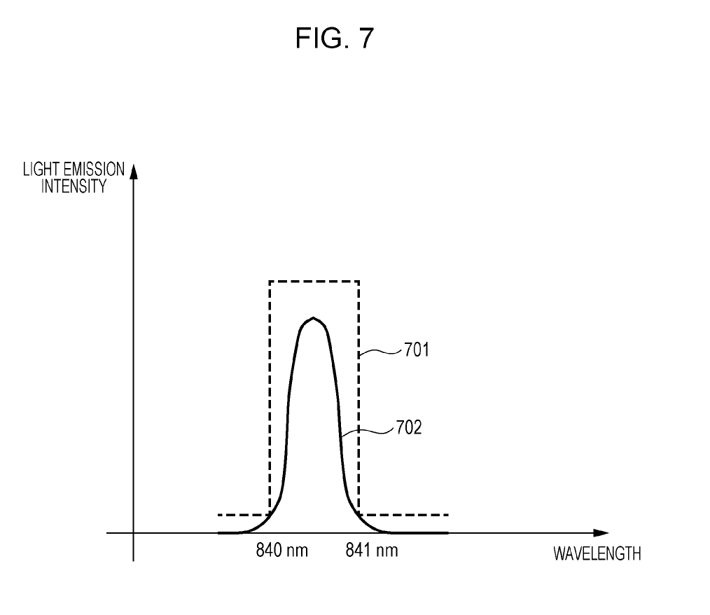

As illustrated in FIG. 7, a spectrum 702 can be obtained by allowing light from the light source unit 407 to pass through a band-pass filter having a known transmittance spectrum 701.

Since the spectrum 702 is a convolution of the instantaneous spectrum in this wavelength band and the transmittance spectrum 701 of the band-pass filter, the instantaneous spectral linewidth in this wavelength band can be obtained using the transmittance spectrum 701 and the spectrum 702.

As the instantaneous linewidth measurement with such a band-pass filter is carried out at some wavelengths between 800 nm and 880 nm, the variation in the coherence length during wavelength sweeping can be found with ease. Preferably, the slit 410 is driven to vary a wavelength selection width or the electro-optical modulation element 403 is driven to control the net gain so that the coherence length during wavelength sweeping stays constant. FIG. 4B illustrates an OCT apparatus configured according to the first exemplary embodiment, and the OCT apparatus is of a Michelson interferometer type. The apparatus illustrated in FIG. 4B employs the light source unit 407 illustrated in FIG. 4A as the light source unit 407.

The light source unit 407 introduces light into the apparatus illustrated in FIG. 4B. The light is split by a coupler 416, and a part of the light is incident on a mirror 419 (e.g., reference mirror) through a collimator 417. Then, the light reflected by the mirror 419 is detected by a photo-detector 421, which is an optical detector.

The other part of the split light is incident on a mirror 420 (e.g., sample) through a collimator 418, and similarly, the light reflected by the mirror 420 (e.g., reflection light from the sample) is detected by the photo-detector 421.

Here, the photo-detector 421 detects an interference spectrum of the reflection light from the mirror 419 and the reflection light from the mirror 420.

The photo-detector 421 sends a signal to a light source control device 422. The light source control device 422 carries out feedback control on the light source unit 407 to control the filter width, the quantity of light from the light source, the driving speed, and so on so that the instantaneous spectral width stays constant.

Then, this interference spectrum is obtained while varying the distance to the mirror 419, for example.

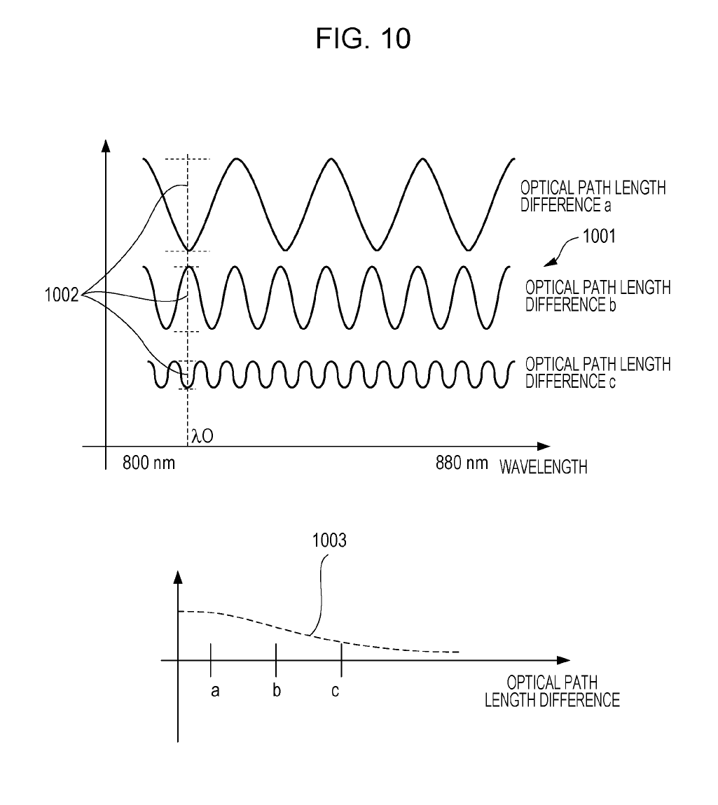

That is, the interference spectrum is obtained while varying an optical path length difference between an optical path length from the coupler 416 to the mirror 419 and an optical path length from the coupler 416 to the mirror 420. Then, a set of interference spectra 1001 corresponding to optical path length differences a to c is obtained (see FIG. 10).

As the optical path length difference increases, the amplitude of the interference spectrum decreases. Amplitudes 1002 of interference signals at a wavelength λ₀ are extracted from the set of interference spectra 1001.

Then, an optical path length difference at which the amplitude of an envelope 1003 of a graph illustrated in FIG. 10 is ½ corresponds to the coherence length and reflects an instantaneous linewidth of the light emitted from the light source unit 407.

It is also preferable that the slit 410 be driven to vary a wavelength selection width or the electro-optical modulation element 403 be driven to control the net gain so that the coherence length during wavelength sweeping stays constant.

### Second Exemplary Embodiment

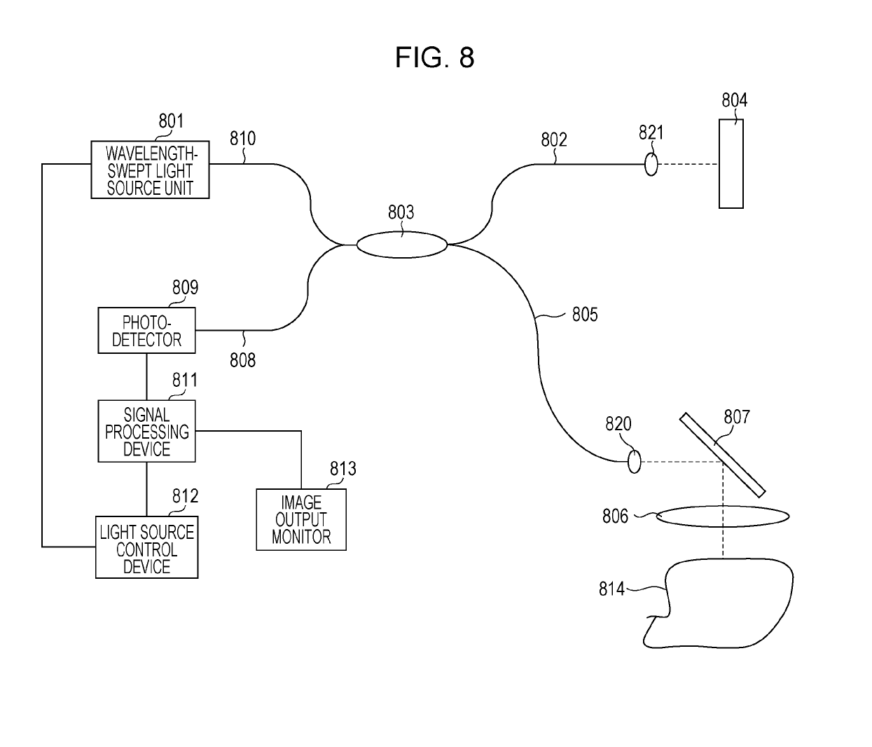

With reference to FIG. 8, an example of an OCT apparatus in which a method for driving a wavelength-swept light source according to an exemplary embodiment of the present invention is employed will be described as a second exemplary embodiment.

The apparatus illustrated in FIG. 8 includes a wavelength-swept light source unit 801 that forms a light source unit, a reference light optical path fiber 802 that forms a reference portion, and a fiber coupler 803 and a reflection mirror 804 that form an interference portion. An inspection light optical path fiber 805, an irradiation condensing optical system 806, and an irradiation spot scanning mirror 807 that form a sample measurement portion are also connected in the apparatus. In addition, a light receiving fiber 808, a photo-detector 809, and an irradiation fiber 810 that form a light detection portion, and a signal processing device 811 and an image output monitor 813 that form an image processing unit are connected in the apparatus.

Then, a light source control device 812 included in the light source unit is further connected to form an optical coherence tomographic imaging apparatus. A sample 814 (object to be inspected) and collimator lenses 820 and 821 are also illustrated in FIG. 8.

In the second exemplary embodiment, the fibers for forming the interference optical system are single-mode fibers, and the fiber coupler is a 3 dB coupler.

The light source control device 812 inputs a control signal to the wavelength-swept light source unit 801.

The light source control device 812 controls the oscillation wavelength, the intensity, and their changes over time, and the coherence length of the wavelength-swept light source 801.

Light emitted from the wavelength-swept light source unit 801 is split by the fiber coupler 803, and the split rays are guided respectively to the reference light optical path fiber 802 and the inspection light optical path fiber 805.

Further, the reflection mirror 804 is provided at a leading end of the reference light optical path fiber 802, and the light reflected by the reflection mirror 804 is guided to the light receiving fiber 808 and reaches the photo-detector 809.

At the same time, the light guided to the inspection light optical path fiber 805 from the fiber coupler 803 is incident on the sample 814, and backscattering light is generated from the interior and the surface of the sample 814. The backscattering light is then focused on the photo-detector 809 via the fiber coupler 803 by the irradiation condensing optical system 806.

The light received by the photo-detector 809 is converted into a spectral signal by the signal processing device 811, which is then subjected to Fourier transform to obtain depth information of the sample 814.

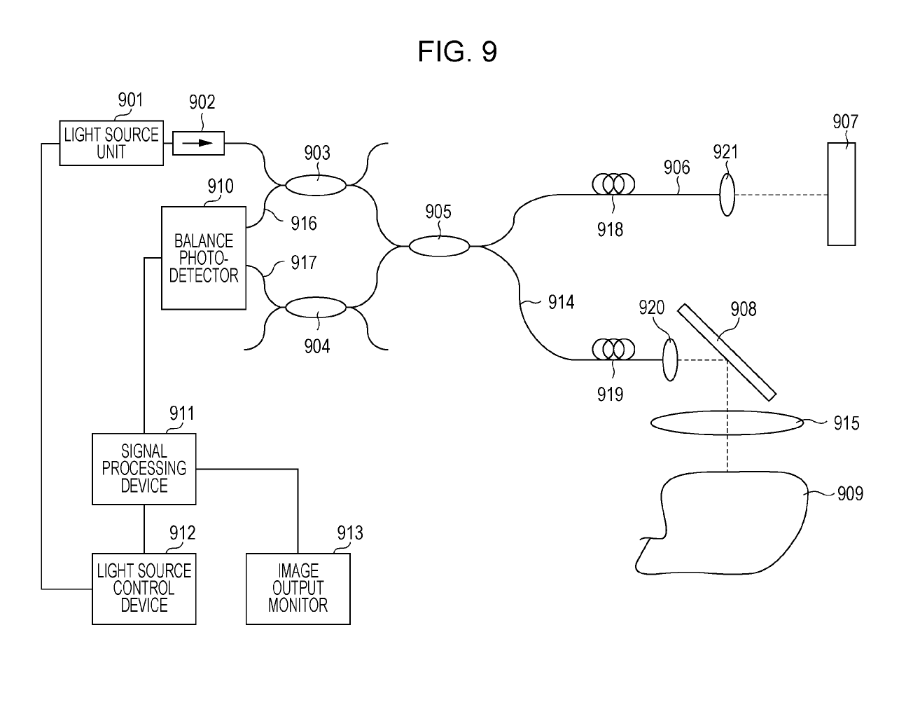

The interferometric system is not limited to the interferometric system illustrated in FIG. 8, and an interference signal may, for example, be obtained using a differential detector. In that case, a configuration illustrated in FIG. 9 may be used.

The apparatus illustrated in FIG. 9 includes a light source unit 901, an isolator 902, a reference light optical path fiber 906 and a polarization controller 918 that form a reference portion, and a fiber coupler 905 and a reflection mirror 907 that form an interference portion. Further, an inspection light optical path fiber 914, a polarization controller 919, an irradiation condensing optical system 915, and an irradiation spot scanning mirror 908 that form a sample measurement portion are also connected in the apparatus.

In addition, fiber couplers 903 and 904, light receiving fibers 916 and 917, and a balance photo-detector 910 that form a light detection unit, and a signal processing device 911 and an image output monitor 913 that form an image processing unit are connected in the apparatus.

A light source control device 912 included in the light source unit is further connected to form an optical coherence tomographic imaging apparatus. A sample 909 (object to be inspected) and collimator lenses 920 and 921 are also illustrated in FIG. 9.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-164477 filed Jul. 25, 2012, which is hereby incorporated by reference herein in its entirety.

What is claimed is:

1. A method for driving a wavelength-swept light source that includes an optical resonator in which an optical gain medium for emitting light, a wavelength selection element for selecting a wavelength of light emitted from the optical resonator, and an optical intensity modulation element for modulating intensity of light within the optical resonator are arranged and that is configured to sweep a wavelength of the emitted light, the method comprising:
driving the optical intensity modulation element and the wavelength selection element in synchronization with each other to keep an instantaneous spectral width of the emitted light constant.

2. The method for driving the wavelength-swept light source according to claim 1, wherein the optical intensity modulation element is driven to satisfy the following formula (2)
c(λ)=α×b(λ)/a(λ) Formula (2)
in which a(λ) is a net gain spectrum of the optical resonator while the optical intensity is not modulated by the optical intensity modulation element, b(λ) is a net gain spectrum of the optical resonator while the optical intensity is modulated by the optical intensity modulation element is, c(λ) is a spectrum of the optical intensity modulation element while the optical intensity modulation element is driven to sweep the wavelength, and α is a positive real number.

3. The method for driving the wavelength-swept light source according to claim 1, wherein the optical intensity modulation element is disposed at a position other than a position between the optical gain medium and the wavelength selection element within the optical resonator.

4. The method for driving the wavelength-swept light source according to claim 1, wherein drive control of the optical intensity modulation element and drive control of the optical gain medium are simultaneously carried out.

5. The method for driving the wavelength-swept light source according to claim 1, wherein the quantity of light from the wavelength-swept light source is kept constant to keep the instantaneous spectral width of the light constant.

6. The method for driving the wavelength-swept light source according to claim 1, wherein the wavelength-swept light source is configured to emit continuous wave light.

7. A wavelength-swept light source, comprising:
an optical resonator including
an optical gain medium configured to emit light,
a wavelength selection element configured to select a wavelength of light emitted from the optical resonator, and
an optical intensity modulation element configured to modulate intensity of light within the optical resonator,
wherein the wavelength-swept light source is configured to sweep a wavelength of the emitted light, and
wherein the optical intensity modulation element and the wavelength selection element are driven in synchronization with each other to keep an instantaneous spectral width of the emitted light constant.

8. An optical coherence tomographic imaging method in which light emitted from a light source unit is split and split rays are radiated onto a sample and a reference reflection portion, respectively, to obtain a tomographic image of the sample based on interference light in which reflection light from the sample and reflection light from the reference reflection portion interfere with each other, the optical coherence tomographic imaging method comprising:
using a wavelength-swept light source that includes, as a light source unit, an optical resonator in which an optical gain medium for emitting light, a wavelength selection element for selecting a wavelength of light emitted from the optical resonator, and an optical intensity modulation element for modulating intensity of light within the optical resonator are arranged and that is configured to sweep a wavelength of the emitted light; and
driving the optical intensity modulation element and the wavelength selection element in synchronization with each other to keep an instantaneous spectral width of the emitted light constant.

9. The optical coherence tomographic imaging method according to claim 8, wherein the instantaneous spectral width of the light is kept constant by driving the optical intensity modulation element such that a coherence length in the optical coherence tomographic imaging stays constant.

10. The optical coherence tomographic imaging method according to claim 8, wherein a wavelength sweeping band in which the wavelength of the emitted light is swept is a value that is equal to or greater than 2×ln(2)/π×λ₀²/Δz, in which Δz is depth-wise resolution in the optical coherence tomographic imaging and λ₀ is a central wavelength of the wavelength band in which the wavelength is swept.
